(19) United States
(12) Patent Application Publication
(10) Pub. No.: US 2005/0124895 A1
(43) Pub. Date: Jun. 9, 2005

(54) Ultrasonic speckle reduction using nonlinear echo combinations

(75) Inventors: Seth Jensen, Bothell, WA (US); Michalakis Averkiou, Kirkland, WA (US)

Correspondence Address: ATL ULTRASOUND, P.O. BOX 3003, 22100 BOTHELL EVERETT HIGHWAY, BOTHELL, WA, 98041-3003, US

(73) Assignee: Koninklijke Philips Electronics N.V.

(21) Appl. No.: 10/984,319

(22) Filed: Nov. 8, 2004

Related U.S. Application Data
(60) Provisional application No. 60/527,538, filed on Dec. 5, 2003.

(51) Int. Cl. A61B 8/06

(52) U.S. Cl. 600/453

(57) ABSTRACT

An ultrasonic imaging apparatus and method are described for imaging nonlinear response objects such as contrast agents and tissue with reduced speckle artifacts. A pulse sequence of two or more pulses of differing amplitude, polarity, and/or phase characteristics is transmitted in each beam direction and an ensemble of echoes is received for each sampled point in the image field. The echoes are combined by different nonlinear signal separation processes and these results are combined to reduce image speckle by a frequency compounding effect. Nonlinear separation techniques which can be used include pulse inversion, power modulation, and combined power modulation/pulse inversion.

This invention claims the benefit of Provisional U.S. Patent Application Ser. No. 60/527,538, filed Dec. 5, 2003.

This invention relates to ultrasonic diagnostic imaging systems and, in particular, to ultrasonic diagnostic imaging systems which reduce image artifacts in nonlinear imaging.

In ultrasonic harmonic imaging, two dimensional (2D) or three dimensional (3D) images are formed by transmitting ultrasound at one frequency (or range of frequencies) and receiving at the higher harmonics of the transmit frequency. These harmonic signals are generated either by scattering from microbubbles of a harmonic contrast agent as described in U.S. Pat. No. 5,833,613 (Averkiou et al.) or by non-linear propagation in tissue (tissue harmonic imaging, or THI) as described in U.S. Pat. No. 5,879,303 (Averkiou et al.) Typically, receive beams are formed predominantly from the second harmonic echo signals, with signals at the transmitted (or “fundamental”) frequency being removed either by filtering or by cancellation techniques such as pulse inversion. See U.S. Pat. No. 5,951,478 (Hwang et al.)

Due to the coherent nature of ultrasonic waves, ultrasound images contain an artifact known as speckle. The speckle artifact results from acoustic interaction of differently phased signals within the medium being imaged. The phenomenon occurs in both fundamental frequency imaging and in harmonic imaging. Two techniques have been developed to reduce the speckle artifact. One technique is known as frequency compounding, and is described in U.S. Pat. No. 4,561,019 (Lizzi et al.) With frequency compounding, echo signals from each point in the image field are separated into different frequency bands, either by transmit frequency modulation or receive frequency separation. The separate frequency bands are detected then combined to reduce the speckle artifact, as the different frequency bands will exhibit different speckle characteristics. Combining the detected signals will average out the speckle artifact, reducing its appearance in the image.

The other technique for reducing speckle is spatial compounding which is described in U.S. Pat. No. 6,210,328 (Robinson et al.) Each point in the image field is insonified from multiple different look directions. The returning echoes from the different look directions are detected and combined to average out the speckle artifact. This reduction in speckle is due to the differing speckle characteristics of ultrasound which has undergone different transmission paths in the medium.

One approach for reducing speckle in harmonic imaging is described in U.S. Pat. No. 6,206,833 (Christopher). In this patent the inventor proposes to form an image which is the sum of both a fundamental frequency image and its corresponding second harmonic image. Since the speckle patterns of the two images are to a certain extent out of phase, the sum image will exhibit reduced speckle. This approach however will contaminate the harmonic image with clutter from the fundamental image, clutter that harmonic imaging eliminates. It would be desirable to be able to reduce speckle in harmonic images without the need for the fundamental signal, which is many dB stronger than the second harmonic signal and is often contaminated with multipath clutter. It would also be desirable to reduce speckle in nonlinear imaging through processing which do not require extensive or complicated bandpass filtering for signal separation.

In accordance with the principles of the present invention, echo signals from transmit sequences of differently modulated transmit signals are combined in different ways to produce nonlinear components with different speckle characteristics. The nonlinear components are combined to produce an image with reduced speckle content. Unwanted linear fundamental frequency components are eliminated by signal processing techniques such as pulse inversion and power modulation and their combinations, obviating the need for bandpass filtering.

In the drawings:

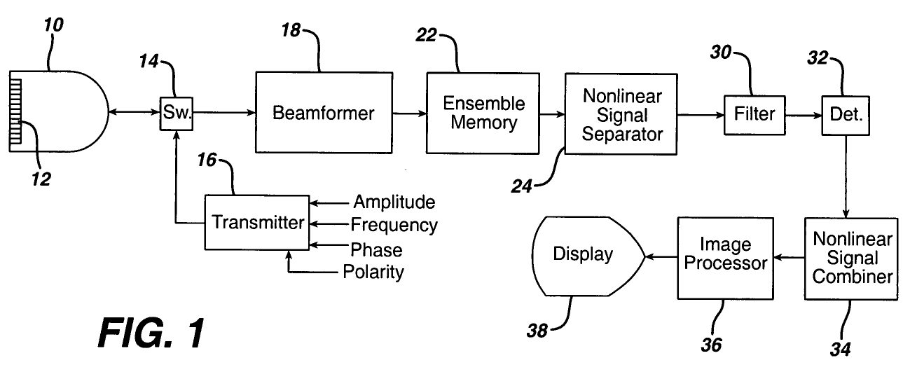

FIG. 1 illustrates in block diagram form an ultrasonic diagnostic imaging system constructed in accordance with the principles of the present invention;

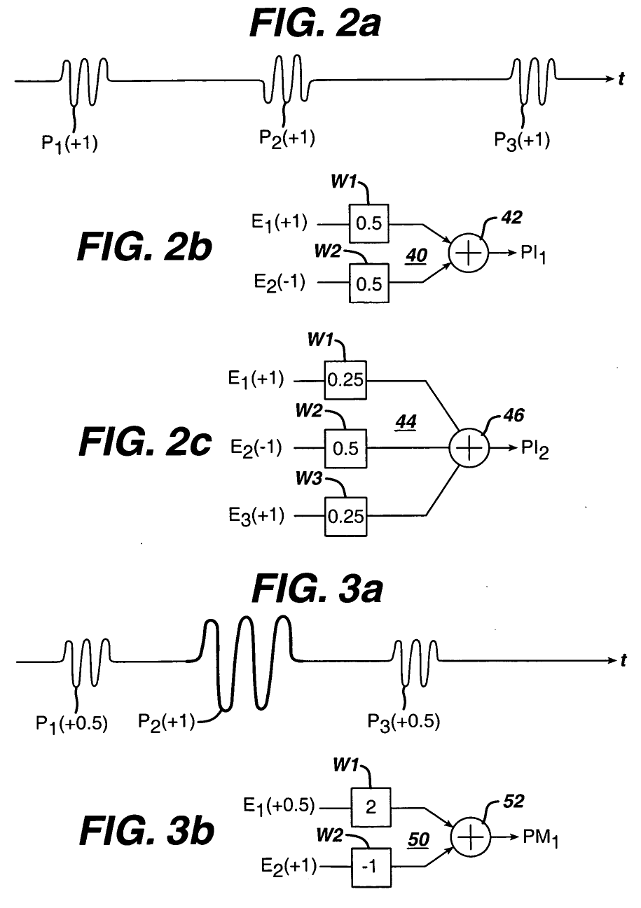

FIGS. 2a, 2b and 2c illustrate a pulse sequence and combining circuits for producing two nonlinear signals by pulse inversion;

FIG. 2d illustrates a frequency spectrum of nonlinear signals separated by pulse inversion;

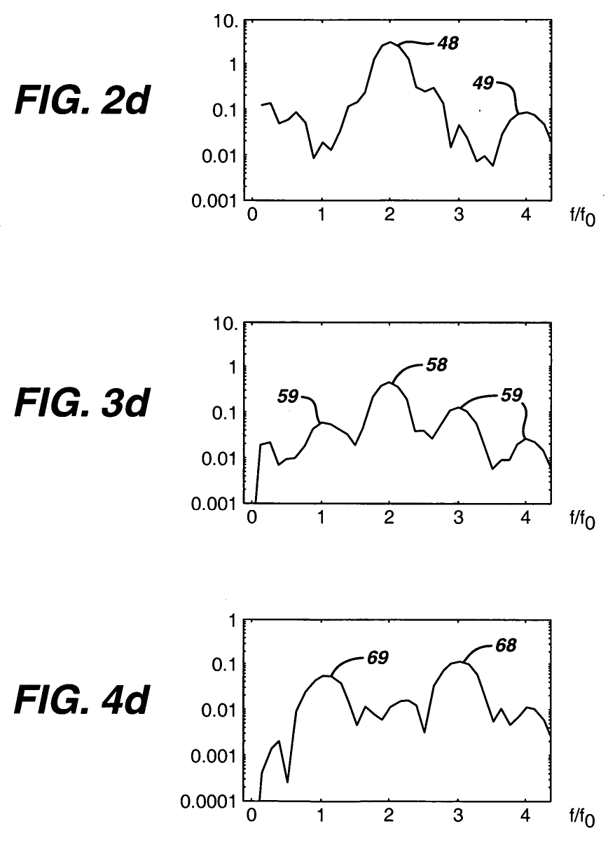

FIGS. 3a, 3b and 3c illustrate a pulse sequence and combining circuits for producing two nonlinear signals by power modulation;

FIG. 3d illustrates a frequency spectrum of nonlinear signals separated by power modulation;

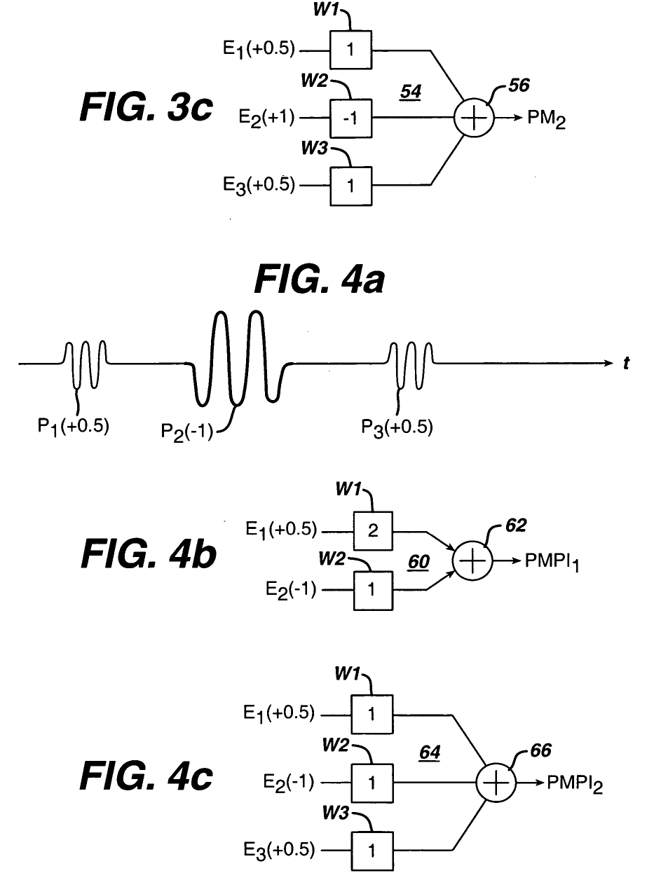

FIGS. 4a, 4b, and 4c illustrate a pulse sequence and combining circuits for producing two nonlinear signals by a combination of power modulation and pulse inversion;

FIG. 4d illustrates a frequency spectrum of nonlinear signals separated by a combination of power modulation and pulse inversion; and

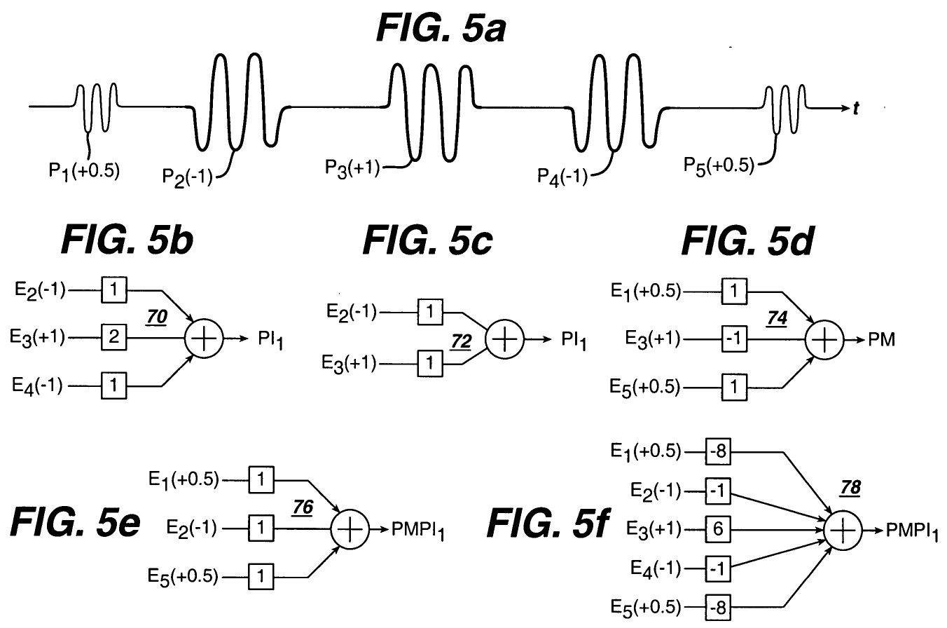

FIGS. 5a-5f illustrate a pulse sequence and combining circuits for producing five different nonlinear signals by pulse inversion, power modulation, and a combination of pulse inversion and power modulation.

Referring first to FIG. 1, an ultrasound system constructed in accordance with the principles of the present invention is shown in block diagram form. This system operates by scanning a two or three dimensional region of the body being imaged with ultrasonic transmit beams. As each beam is transmitted along its steered path through the body, the beam returns echo signals with linear and nonlinear (fundamental and harmonic) components corresponding to the transmitted frequency components. The transmit signals are modulated by the nonlinear effects of the tissue through which the beam passes or the nonlinear response of a contrast agent microbubble encountered by the beam, thereby generating echo signals with harmonic components.

The ultrasound system of FIG. 1 utilizes a transmitter 16 which transmits waves or pulses of a selected modulation characteristic in a desired beam direction for the return of harmonic echo components from scatterers within the body. The transmitter is responsive to a number of control parameters which determine the characteristics of the transmit beams as shown in the drawing, including the frequency components of the transmit beam, their relative intensities or amplitudes, and the phase or polarity of the transmit signals. The transmitter is coupled by a transmit/receive switch 14 to the elements of an array transducer 12 of a scanhead 10. The array transducer can be a one dimensional array for planar (two dimensional) imaging or a two dimensional array for two dimensional or volumetric (three dimensional) imaging.

The transducer array 12 receives echoes from the body containing linear and harmonic (nonlinear) frequency components which are within the transducer passband. These echo signals are coupled by the switch 14 to a beamformer 18 which appropriately delays echo signals from the different transducer elements then combines them to form a sequence of linear and harmonic signals along the beam from shallow to deeper depths. Preferably the beamformer is a digital beamformer operating on digitized echo signals to produce a sequence of discrete coherent digital echo signals from a near field to a far field depth of field. The beamformer may be a multiline beamformer which produces two or more sequences of echo signals along multiple spatially distinct receive scanlines in response to a single transmit beam, which is particularly useful for 3D imaging. The beamformed echo signals are coupled to an ensemble memory 22

In accordance with the principles of the present invention, multiple waves or pulses are transmitted in each beam direction using different modulation techniques, resulting in the reception of multiple echoes for each scanned point in the image field. The echoes corresponding to a common spatial location are referred to herein as an ensemble of echoes, and are stored in the ensemble memory 22, from which they can be retrieved and processed together. The echoes of an ensemble are combined in various ways as described more fully below by the nonlinear signal separator 24 to produce the desired nonlinear or harmonic signals. The separated signals are filtered by a filter 30 to further remove unwanted frequency components, then subjected to B mode or Doppler detection by a detector 32. The detected signals are coupled to a nonlinear signal combiner 34 to reduce image speckle content, as described more fully below. The signals are then processed for the formation of two dimensional, three dimensional, spectral, parametric, or other desired image in image processor 36, and the image is then displayed on a display 38.

FIG. 2a illustrates a sequence of differently modulated transmit pulses (“P”) which are transmitted along a beam direction. The subscript of each pulse P indicates the position of the pulse in the sequence. These subscripts are only necessary to clarify the following description, as the pulses in a sequence can be transmitted in any order. The parenthetical of each pulse P indicates the relative amplitude and phase or polarity of a given pulse. In the sequence of FIG. 2a the first transmit pulse P₁(+1) is seen to have an amplitude of “one”, and a positive phase or polarity relative to other pulses in the sequence. The second pulse P₂(−1) also has an amplitude of one but a phase or polarity which is the inverse of the first pulse. The third pulse in the time sequence is seen to have an amplitude of one and a positive phase or polarity. Thus it is seen that the second pulse is differently modulated (in phase or polarity) relative to the other two pulses in the sequence.

Echoes are received along the beam direction in response to each pulse, resulting in an ensemble of three echoes (“E”) at each sample point of the beam. The echoes of the ensembles are combined in different ways by the nonlinear signal separator 24. In the signal separator circuit 40 of FIG. 2a, echo E₁(+1) from the first pulse is weighted by a weight of 0.5 in weighting circuit W1 and applied to a summer 42. Echo E₂(−1) from the second pulse is weighted by a weight of 0.5 in weighting circuit W2 and also applied to summer 42, where the two weighted echoes are combined. Since the two echoes are from pulses of opposite phase or polarity and of equal amplitude, the equally weighted combining of the echoes results in cancellation of fundamental signal components of the echoes returned from stationary targets and reinforcement of the nonlinear (second and higher order even harmonic) signal components, a phenomenon known in the art as pulse inversion. See U.S. Pat. Nos. 5,706,819 (Hwang) and 5,951,478 (Hwang et al.) The resultant nonlinear signals are denoted as PI₁, indicating that these nonlinear signals were separated by a first pulse inversion combination. In the case of moving scatterers such as contrast agent microbubbles pulse inversion processing also produces signals from the motion of microbubbles that lie mostly in the fundamental frequency band.

FIG. 2c illustrates a second signal separator circuit 44 which also separates nonlinear signals from fundamental frequency components by the pulse inversion technique. The echo E₁(+1) is weighted by a weighting factor of 0.25 in weighting circuit W1 and applied to a summer or combiner 46. Echo E₂(−1) is weighted by a weighting factor of 0.5 in weighting circuit W2 and also applied to summer 46. Echo E₃(+1) from the third pulse is weighted by a weighting factor of 0.25 in weighting circuit W3 and also applied to summer 46. Like the weights of the signal separator circuit 40, the weights of this signal separator circuit are also normalized to a sum of one. The one-quarter weightings of the echoes from the positive phase or polarity pulses when combined with the one-half weighting of the echo from the negative phase or polarity pulse P₂(−1) results in pulse inversion separation of nonlinear signals PI₂ with suppression of the fundamental frequency components of the echoes from stationary targets and reinforcement of the nonlinear (second harmonic) signal components. Thus it is seen that the two signal separator circuits both produce nonlinear signal components and flow components from a given point in an image field but by different pulse inversion signal combinations. The different receive weights cause PI₁ and PI₂ to detect different velocities of moving scatterers.

FIG. 2d illustrates a typical frequency spectrum of the signals separated by pulse inversion (PI₁ or PI₂). This frequency spectrum is seen to be dominated by a major peak response 48 at the second harmonic and a lesser peak 49 at the fourth harmonic.

FIG. 3a illustrates another pulse sequence in which the pulses are differently modulated in amplitude. The first and third pulses P₁(+0.5) and P₃(+0.5) are each seen to have an amplitude of one-half relative to the amplitude of the second pulse P₂(+1). All of the pulses are seen to exhibit the same (positive) phase or polarity. Each three-echo ensemble is then processed as shown by the signal separator circuits 50 and 54. Circuit 50 weights an echo E₁(+0.5) from the first pulse by a weight of 2 in weighting circuit W1 and applies the weighted echo to the summer 52. The echo E₂(+1) from the second pulse is weighted by a weight of −1 in weighting circuit W2 and applied to the summer 52. The combined weightings of the echoes from the differently amplitude modulated (power modulated) pulses results in separation of nonlinear signal components PM₁ by the power modulation technique. See U.S. Pat. No. 5,577,505 (Brock Fisher et al.)

In FIG. 3c nonlinear components are again separated by the power modulation technique, but this time using three echo signals. The echo E₁(+0.5) is weighted by a weight of 1 in weighting circuit W1 and applied to summer 56. The echo E₂(+1) is weighted by a weight of −1 and applied to the summer 56. The echo E₃(+0.5) is weighted by a weight of 1 in weighting circuit W3 and applied to the summer 56. The combination of the three weighted, differently amplitude modulated signals results in another nonlinear signal PM₂ separated by a different power modulation combination of echoes. The two nonlinear signals PM₁ and PM₂ will exhibit a frequency spectrum such as that illustrated in FIG. 3d, which is seen to be characterized by a major response peak 58 at the second harmonic and lesser peaks 59 at the fundamental (first) and third and fourth harmonics.

FIG. 4a illustrates a sequence of transmit pulses for a given beam direction which are differently modulated in both amplitude and phase or polarity. The first and third pulses P₁(+0.5) and P₃(+0.5) are both seen to have a positive phase or polarity and a relative amplitude of one-half. The second pulse P₁(−1) is seen to exhibit an inverse phase or polarity and an amplitude of one, which is twice that of the first and third pulses. Various echo combinations can be formed to separate nonlinear or harmonic components by the combined technique referred to herein as power modulation/pulse inversion (PMPI). FIG. 4b shows a signal separator circuit 60 in which echo E₁(+0.5) is weighted by a weight of 2 and combined in summer 62 with echo E₂(−1) which is weighted by a weight of 1. The amplitude difference of the two echoes is equalized by the weighting factors and the differently phased echoes combine to produce a first nonlinear signal PMPI₁ by a combination of pulse inversion and power modulation. In FIG. 4c the three echoes of an ensemble are each weighted by a weight of one and combined by summer 66 of signal separator circuit 64 to produce a second nonlinear signal PMPI₂. The signals produced by the different summations of PMPI modulated signals will produce a frequency spectrum such as that shown in FIG. 4d, which is seen to be characterized by a major response peak 68 at the third harmonic and a lesser peak 69 at the fundamental (first) harmonic.

In accordance with the principles of the present invention, echoes returned from microbubbles which have been differently processed by the PI, PM and PMPI techniques described above to yield signals with differing spectra such as those shown in FIGS. 2d, 3d, and 4d are combined to reduce the speckle content of an ultrasonic contrast image. FIGS. 5a-5c illustrate an embodiment of the present invention in which a transmit sequence of five pulses is employed in each beam direction, resulting in ensembles of five echoes for each sample point in the image field. As FIG. 5a illustrates, the first and fifth pulses P₁(+0.5) and P₅(+0.5) both exhibit the same phase or polarity as well as the same amplitude. The other three pulses P₂(−1), P₃(+1) and P₄(−1) all have an amplitude which is twice that of the first and last pulses. The third pulse exhibits the same phase or polarity as the first and last pulses and the second and fourth pulses are of an inverse (opposite) phase or polarity.

In FIGS. 5b-5f, echoes from the resulting five-echo ensembles are combined for harmonic separation in five different ways, using pulse inversion (PI), power modulation (PM), and power modulation/pulse inversion (PMPI). In the signal separator circuit 70 of FIG. 5b echoes from the second, third, and fourth pulses are weighted by respective weights of 1, 2, and 1 and combined to separate nonlinear signal components PI₁ by pulse inversion. These signal components include second and fourth harmonic components of the transmitted fundamental frequencies, of which the second harmonic is the dominant signal (see FIG. 2d). In the signal separator circuit 72 of FIG. 5c echoes from the second and third pulses are weighted equally and combined, again separating nonlinear signal components PI₂ by pulse inversion. These signal components also include second and fourth harmonic components of the transmitted fundamental frequencies, of which the second harmonic is the dominant signal. In the signal separator circuit 74 of FIG. 5d echoes from the first, third and fifth pulses are combined with respective weights of 1, −1, and 1, resulting in the production of nonlinear signal components PM by power modulation. The separated signal components include the first, second, third and fourth harmonics, of which the second harmonic is the greatest contributor and the first, third and fourth harmonics are lesser contributors (see FIG. 3d). In FIG. 5e echoes from the first, second and fifth pulses are equally weighted and combined by a signal separator circuit 76 to produce nonlinear signal components PMPI₁ by the combined PMPI technique. These signal components include the first through the fourth harmonics, of which the first and third harmonics are the major contributors (see FIG. 4d). In FIG. 5f a signal separator circuit 78 operates on echoes from all five pulses. Echoes from the first and last pulses, which exhibit the lesser amplitudes, are weighted by −8. Echoes from the second, third, and fourth pulses are weighted by weights of −1, 6, and −1, respectively. This combination will result in nonlinear signal components PMPI₂ by the combined technique, and include the first, second, and third harmonics, of which the first and third are predominant.

It is seen from the preceding examples that the various separated nonlinear signals are dominated by varying frequency components. Thus, the signals have differing frequency content. As a consequence, when these five signals are combined by the nonlinear signal combiner 34, speckle reduction will occur by a frequency compounding effect.

In a constructed embodiment of the present invention it is often preferable to combine the echo signals, not with dedicated hardware separator circuits, but mathematically in a matrix operation. Using the previous five-pulse embodiment as an example, the transmit matrix would be of the form

$$[ \begin{matrix}0.5 , & - 1 , & 1 , & - 1 , & 0.5\end{matrix} ]$$

and the receive matrix would be of the form

$$[ \begin{matrix}0 , & 1 , & 2 , & 1 , & 0 \\ 0 , & 1 , & 1 , & 0 , & 0 \\ 1 , & 0 , & - 1 , & 0 , & 1 \\ 1 , & 1 , & 0 , & 0 , & 1 \\ - 8 & - 1 & 6 & - 1 & - 8\end{matrix} ]$$

The desired signals are produced by multiplication of matrices of this form. Since the different combining techniques extract different nonlinear components, the combination of their results will produce a frequency compounded image with reduced image speckle.

It will be understood that weights other than 0.5 and 1 may be used, and phases other than 0 and p may be used. The specific transmit sequence used will be determined at least in part by the desired harmonic content to be obtained. The relative content of the different harmonics introduced according to the receive processing may be scaled so that different effects are emphasized. For the matrix representation above a different scaling may be applied to various rows of the matrix. If for example it is desired to emphasize the relative effect of pulse inversion by a factor of two, then the above matrix would become

$$[ \begin{matrix}0 , & 2 , & 4 , & 2 , & 0 \\ 0 , & 2 , & 2 , & 0 , & 0 \\ 1 , & 0 , & - 1 , & 0 , & 1 \\ 1 , & 1 , & 0 , & 0 , & 1 \\ - 8 & - 1 & 6 & - 1 & - 8\end{matrix} ]$$

1. An ultrasonic diagnostic imaging system for nonlinear imaging comprising:
a transmitter which acts to transmit sequences of differently modulated ultrasonic signals over an image field;
a receiver which receives echo signals in response to the transmit sequences;
a storage device which stores echo ensembles corresponding to the transmit sequences;
a nonlinear signal separator, responsive to the echo ensembles, which combines echoes from an ensemble in different ways to produce nonlinear signals; and
a nonlinear signal combiner, responsive to the nonlinear signals, which combines differently produced nonlinear signals corresponding to an image location to produce a speckle-reduced signal component corresponding to the image location.

2. The ultrasonic diagnostic imaging system of claim 1, wherein the nonlinear signal separator acts to produce nonlinear signals by at least one of the techniques of pulse inversion, power modulation, or combined power modulation/pulse inversion.

3. The ultrasonic diagnostic imaging system of claim 2, wherein the nonlinear signal separator acts to produce nonlinear signals of differing harmonic content.

4. The ultrasonic diagnostic imaging system of claim 1, further comprising a detector, coupled to the nonlinear signal separator, which acts to detect the nonlinear signals.

5. The ultrasonic diagnostic imaging system of claim 4, wherein the detector acts to detect at least one of B mode or Doppler signals.

6. The ultrasonic diagnostic imaging system of claim 1, wherein the transmitter acts to differently modulate signals in at least one of amplitude, phase, or polarity.

7. A method for producing a speckle-reduced harmonic image comprising:
transmitting sequences of differently modulated ultrasonic signals over an image field;
receiving ensembles of echoes in response to transmitted sequences;
combining echoes of an ensemble in different ways to produce nonlinear signal components; and
combining nonlinear signal components relating to a common spatial image location to produce speckle-reduced harmonic signals.

8. The method of claim 7, wherein transmitting further comprises transmitting sequences of pulses which are differently modulated in at least one of amplitude, phase, or polarity.

9. The method of claim 7, wherein combining echoes of an ensemble further comprises extracting different nonlinear signal components from an ensemble of echoes.

10. The method of claim 7, further comprising producing an image using the speckle-reduced harmonic signals.

11. The method of claim 10, wherein producing an image further comprises producing a B mode image.

12. The method of claim 10, wherein producing an image further comprises producing a Doppler image.

13. The method of claim 7, further comprising detecting the nonlinear signal components.

14. A method for producing a speckle-reduced harmonic image comprising:
transmitting a plurality of differently modulated transmit signals to spatial locations in an image field;
combining different pluralities of echoes corresponding to a common spatial location to extract different signal components corresponding to the common spatial location; and
combining the different signal components to produce a signal corresponding to the common spatial location with reduced speckle content.

15. The method of claim 14, wherein combining further comprises at least one of the nonlinear signal processing techniques of pulse inversion, power modulation, or power modulation/pulse inversion.

16. The method of claim 14, wherein combining different pluralities of echoes further comprises extracting different nonlinear components.

17. The method of claim 14, wherein combining different pluralities of echoes further comprises extracting different harmonic signal components.

18. The method of claim 14, further comprising detecting the different signal components.

19. A method for producing a speckle-reduced harmonic image comprising:
transmitting a plurality of differently modulated transmit signals to spatial locations in an image field;
combining different pluralities of echoes corresponding to a common spatial location to extract signals with different harmonic components corresponding to the common spatial location; and
combining signals with different harmonic components to produce a signal corresponding to the common spatial location with reduced speckle content.

20. The method of claim 19, wherein combining further comprises at least one of the nonlinear signal processing techniques of pulse inversion, power modulation, or power modulation/pulse inversion.

21. The method of claim 19, further comprising detecting the signals with different harmonic components.
